(19) United States
(12) Patent Application Publication
(10) Pub. No.: US 2014/0102957 A1
(43) Pub. Date: Apr. 17, 2014

(54) PATIENT SCALES WITH CAMERA-SUPPORTED MONITORING AND A DIALYSIS THERAPY SYSTEM WITH CAMERA-CONTROLLED WEIGHING PROCESS

(71) Applicant: B. BRAUN AVITUM AG, MELSUNGEN (DE)

(72) Inventors: BJOERN BROEKER, STAUFENBERG (DE); KAI-UWE RITTER, MELSUNGEN (DE)

(21) Appl. No.: 14/053,202

(22) Filed: Oct. 14, 2013

(30) Foreign Application Priority Data
Oct. 16, 2012 (DE) 10 2012 109 861.1

(51) Int. Cl. A61B 5/00; A61M 1/14; G01G 23/35

(52) U.S. Cl. CPC A61B 5/0013; A61B 5/0077; G01G 23/35; A61B 5/4839; A61B 5/0022; A61M 1/14
USPC 210/85; 600/476

(57) ABSTRACT

A weight detection device is disclosed for determining a patient's weight, for dialysis treatment, that includes patient scales arranged to carry out a weighing procedure to detect the actual weight of a patient, preferably a network connection arranged to connect a component of the weight detection device, generating information and/or data related to the determining of the patient's weight and/or to the weighing procedure, to a data network, an output device arranged to output the actual weight of the patient, and an image information recording device that includes a field of view directed to the patient scales for the visual detection of weighing procedures arranged to record the course of a weighing procedure such that a cause of a change in the actual weight occurring in the course of dialysis therapy can be determined from a recording of the weighing procedure.

### CROSS REFERENCE TO RELATED APPLICATIONS

This application claims priority to German application DE 10 2012 109 861.1 filed Oct. 16, 2012, the contents of such application being incorporated by reference herein.

### FIELD OF THE INVENTION

The invention relates to a weight detection device comprising patient scales and the video-supported monitoring of a weighing process by means of a camera, in the context of dialysis therapy, and to a dialysis therapy system with a camera-controlled weighing process, and relates in particular to patient scales as a component of the weight detection device, which are designed for recording the weighing procedure of a patient in the context of dialysis treatment, in connection with a camera, as well as to a system comprising such scales and used for dialysis therapy for renal insufficiency.

### BACKGROUND OF THE INVENTION

Dialysis therapies or dialysis treatments often require carrying out frequent weiging procedures before, during, and after the actual dialysis. Dialysis patients have insufficient function of the natural kidney. Its tasks, for instance the removal of uremic toxines and the expulsion of excess body water, have to be taken over by an artificial kidney (a dialyzer). In one embodiment of the artificial kidney, the latter presses out plasma water of the patient via a dialyzer membrane during its blood cleaning procedure. Such a process of pressing out plasma water, or such removal of water by filtration of the blood through a sieve membrane, at a pressure from 0 to 1 bar, accompanied by attendant effects (convection) for selective detoxification, is referred to as ultrafiltration. By means of ultrafiltration, it is possible to remove or wash out particles of molecular substances of average size (ranging from approximately 0.1 to 0.05 μm), such as endotoxines, viruses, colloids, proteins, and enzymes, from the blood of a patient, and to clean it in this way. As this type of cleaning is effected outside the body of a patient, the body loses water and for instance electrolytes in this process, which have to be substituted. Further, large filtrate quantities may arise depending on the permeability of the filters, which go beyond the necessary volume withdrawal of the patient. The substitution of liquid of a physiological composition corresponding to the filtrate quantity is therefore required in order to stabilize the fluid balance of the patient.

In contrast to people with healthy kidneys, the ability to regulate the water balance in the body is impaired to more or less an extent in people suffering from renal diseases, so that too much water is retained in the body. This results in a surplus load on an organism as one of the main factors which represent increasing complications and a shortened life expectancy for the patient. Therefore, one of the most important tasks of dialysis treatment is to replace the missing regulation of the water balance as best as possible. Usually, what is known as dry weight is defined as the therapeutic goal in dialysis treatments. One proposed definition of dry weight uses an estimated value for the same weight the dialysis patient would have if he/she had no renal disease. From the medical point of view, the dry weight is referred to as the weight by the end of dialysis, at which the patient does not show any indications of overhydration, is free from symptoms, feels good, and continues to have normal blood pressure until the next dialysis.

For determining the dry weight, which as such cannot be easily specified by the result of an objective procedure, procedures as precise as possible for weighing the patient are indispensable to enable assessment of the ultrafiltration quantity such that the dry weight as the target weight is reached as precisely as possible. From observations, however, it is known that inexplicable and undesired deviations from the ultrafiltration quantity during a weighing procedure or between individual weighing procedures occur again and again. In such cases, in order to be able to prevent dialysis apparatuses causing such deviations, said apparatuses must be taken out of operation in a time-consuming and expensive manner and their accuracy checked in a simulated treatment. In doing so, it turns out frequently that the machine functions satisfactorily.

In that case, it may happen that the cause of a deviation is due to reasons which cannot be understood yet. Examples of a variety of such causes include the fact that a patient has relieved him/herself between weighing procedures, which has not been noted properly, that a patient intentionally gives an incorrect weight with the aim of having a larger quantity of liquid extracted in order to be able to drink more than the allowed amount afterward, that the intake of food and/or beverages has not been logged, that an infusion administered during the treatment has not been measured exactly and/or has not been logged, that a patient does not wear the same clothing in two weighing procedures to be assigned to each other; that a patient uses different wheelchairs of differing weights in two weighing procedures to be assigned to each other, that any items (a bag, a cell phone or the like) have been removed/not removed for a short time during weighing, that the wheelchair which was used did not have an optimum position, or that a patient supports him/herself during a weighing procedure.

FIG. 4 shows for example a simplified illustration of the known course of a weighing procedure, using a scheme of logging detected data on paper. Specifically, the illustrated time schedule shows in chronological sequence the steps or activities, which are to be carried out by the patient and the personnel or staff of a dialysis ward during the procedure of weighing a dialysis patient in the context of a therapy process with preliminary weighing prior to therapy, on the basis of the data determined during the weighing procedure, and with a repeated, monitored weighing process at the end of the therapy. In so far as activities and/or steps must be carried out in parallel and/or simultaneously, the numbering of the individual steps is not necessarily to be understood as a sequence or succession, but for the sake of convenience may only represent an indication of reference numerals for clear assignment.

The designations such as “activity patient”, “objective”, and “document” during the time schedule of the weighing procedure, indicated in FIG. 4 from left to right and one below the other in the individual lines, may include variable assignment between activities, patients and/or staff. To give an example, a patient may carry out a logging activity or the staff may carry out said logging activity.

According to FIG. 4, a dialysis therapy process begins with the arrival of a patient at the dialysis center in step S410. In step S411, he/she takes off his/her clothing at least partially, for instance a jacket and any baggage possibly carried along by him/her, in order to obtain a defined starting situation; this is usually performed in a predetermined manner. The weighing procedure occurs in step S412, during which the current actual weight of the patient is determined in step S413. In doing so, the patient is allowed to weigh him/herself, or he/she is invited to be weighed by the staff. In step S414, said actual weight is entered in a weighing protocol by the staff of the dialysis ward, for example. The weighing protocol is continued during dialysis therapy and may include several weighing procedures carried out in its context. Further, the process of logging therapy data and hence weighing procedures is performed over a number of dialysis treatments occurring in separate periods of time. Having determined the actual weight of the patient in step S413, said weight is used as a preliminary measure for dialysis treatment to determine a target ultrafiltration quantity or a current, actual dry weight in step S415. Subsequently, in step S416, therapy data or treatment parameters based thereon are entered in a dialysis apparatus or transferred to it by the staff, and on the basis of said therapy data the dialysis treatment is carried out with the defined or prescribed therapy data in step S417 after the patient has been connected to the dialysis apparatus. The course or the progress of the therapy is logged by the staff in step S418, as simultaneously or as in parallel with the previous step as possible. Following the end of dialysis therapy, the patient is disconnected from the dialysis apparatus and again weighed in step S419. Once more, the actual weight and the actual dry weight of the patient are determined during the weighing procedure in step S420, as simultaneously or as in parallel with the previous step as possible, and are entered in step S421 in the context of a documentation of the treatment, in the continued protocol and/or entered in a databse, for instance an electronic patient database or patient file, in step S423. After the weighing procedure, the patient is allowed to put on his/her clothes in step S422 and leave the dialysis center in step S424.

As in the purely manual documentation carried out during the therapy process according to FIG. 4, the staff has, apart from taking care of the patients, the task of carrying out the documentation, i.e. completing paper documents and/or transfering the documentation to other electronic devices, it is difficult for the staff to remember details because of the large number of operations concentrated into a narrow timespan. This is why manual documentation of the therapy by staff and/or the patient itself is subject, inter alia, to the possibilities of failures and attributable deviations, which have been mentioned above by way of example.

A system intended for monitoring and comprising a database, known from the prior art, comes from the applicant herself. This monitoring system is a software for the transparent imaging and control of numerous operations in dialysis treatment. The generated data delivered by connected dialysis units, analyzers (e.g. for blood gas analysis), and patient scales are transferred automatically to the monitoring system and saved. This is the place where they are visualized and available for processing. Due to the bidirectional data transfer between the known monitoring system and the connected dialysis units, the data records are also consistent and can be retrieved during treatment.

It is true that known devices in this field are able to provide a certain support for the operational sequence in a dialysis ward by networking individual systems such as machines, scales, and medication. However, they are not capable of giving understandable information about any deviations between a patient's weights and/or ultrafiltration quantities occurring while weighing the patients.

### SUMMARY OF THE INVENTION

The invention relates to the problem of providing patient scales and a dialysis therapy system including such patient scales, which enable a weighing procedure for determining the weight of a patient and required for the dialysis treatment of the patient with respect to any causes of occurring deviations to be carried out in a comprehensible manner, and which enable a reduction in occurring deviations and ultrafiltration quantities to be determined and considered during dialysis treatment in a more precise way.

This problem is solved according to aspects of the invention by the combination of features of claim 1, alternatively by the combination of features of claim 10. Advantageous further developments of the invention are the subject-matter of the enclosed sub-claims.

Thus, the basic idea of the invention is to detect and to evaluate in full the procedures “weighing prior to and after dialysis, as well as during the actual dialysis treatment” per video technology. A device for the improved monitoring of the patient's weight before, during, and after the dialysis procedure is suggested, with videocameras, optionally, networked ones, being arranged on the scales and/or on the dialysis apparatus, and/or in their vicinity.

In particular, the patient scales forming part of a weight determination device and/or weight detection device can be observed with at least one camera. It is preferred that the patient scales and the camera are connected via a network to a data transfer and evaluation system which as a part of a dialysis therapy system forms a monitoring system. The data transfer and evaluation system supports the display and storage of the entire operations as a video recording or in images. The data transfer and evaluation system is able to support the automated evaluation of these recordings. The camera may be installed in the patient scales or in a dialysis apparatus, or in their vicinity with a view onto room areas and/or onto components of the apparatus relevant to a weighing procedure, such as a display device for example, or may be provided in another suitable place. Weighing procedures are logged by means of images or as video recording(s) and saved in the monitoring system. A patient can be identified on the basis of a combination of weight and face recognition. At least, it can be ensured that there is a match between the patient card and the patient. It is possible to identify the nurse who has carried out the weighing procedure. When weighing “after dialysis”, a monitor may show images of the weighing procedure “prior to dialysis”, in order to avoid mistakes. If there are any inexplicable differences in weight between before and after, the weighing procedures can be compared on a monitor or automatically per image matching. Wheelchairs and the like can be identified via a barcode or color coding, and a deviation or offset can be automatically calculated. To this end, the weight of the wheelchairs can be deposited in a database. The start of a weighing procedure may be initiated in gesture-controlled manner. Gesture-based control may extend to the entire course of a weighing procedure which is monitored in a camera-supported fashion.

Further, a dialysis apparatus may be provided, which in cooperation with the patient scales may constitute at least part of a weight monitoring system, and comprises at least one camera mounted thereon or in another suitable place. The patient is observed by means of the camera during a dialysis procedure. Preferably, the camera is connected to a monitoring system via a network. The entire procedure can then be saved in the monitoring system. Starting from saved data, program routines analyze any movements of the patient and detect any intake of food and/or liquids. It is possible to carry out the quality management of measures during dialysis, for instance as to whether drugs have been administered and also been taken etc. An image from the camera may also be used as a direct broadcast (“online” or live transmission), in order to look for a patient. Further, an image from the camera may also be used for a video-conferencing connection to a patient. Moreover, the system may detect a patient walking to washrooms, toilets, and the like and determine whether a corresponding weighing procedure is being carried out or has been carried out.

As a whole, advantages are achieved according to aspects of the invention to the effect that the video recordings are (or can be) evaluated (only) for inexplicable deviations in the weight before and after dialysis, and that relevant scenes are (or can be) filtered out by means of automatic evaluation of the video sequences, and the staff is able to assess these and to evaluate and correct the possible misbehavior of a patient. Patients showing regular deviations can be stored in the system, so that based thereon the system can provide an indication that a weighing procedure must not be carried out independently by a patient him/herself or can be carried out only with support or in the presence of staff.

To this end, a system for an improved monitoring of the weight of a patient before, during, and after the dialysis procedure is suggested, using networked video cameras mounted on scales and/or on dialysis apparatus or in a room or area with a view onto portions of the former delivering the information to be detected.

Thus, the object is achieved by a weight detection device for determining a patient's weight, for dialysis treatment, characterized by patient scales arranged to carry out a weighing procedure and thus detect the actual weight of a patient standing on the scales; optionally, a network connection arranged to connect at least one component of the weight detection device, generating information and/or data related to the determining of the patient's weight and/or to the weighing procedure, to a data network; an output device arranged to output the actual weight of the patient detected on the patient scales in a readable manner and/or in a predetermined data format to the data network, or as an alternative/in addition to that, a possibly erasable data memory; and at least one image information recording device which comprises a field of view directed to the patient scales for the visual detection of weighing procedures and is arranged to record the course of a weighing procedure within the field of view in such a manner that the cause of a change in the actual weight occurring in the course of dialysis therapy can be determined from at least one appropriate recording of at least one weighing procedure.

It is advantageous here if the image information recording device is a camera, preferably with connection to the data network, which is installed in the patient scales.

The advantages of such a camera, which basically is able to record in an analog and/or digital manner, but preferably in a digital manner, are, among other things, the fact that the camera characteristics, for instance with respect to the required resolution, an appropriate telephoto lens range and the scalability (enlarging/reducing) of sections of a recording, a generated video format and/or image format and the like, can be well adapted to the circumstances and requirements of the patient scales and integrated into them, and when the patient scales are moved, the camera is moved together with them in a fixed manner and the adjustment procedures can be reduced to a minimum after moving.

Alternatively, the image information recording device may be a camera, preferably comprising connection to a data network, and being arranged outside the patient scales on a dialysis apparatus.

Here, the advantages are, among other things, that the patient scales can be moved within a pre-adjusted visual range of the camera, for instance if patients have to be supported by staff and the scales is to be moved for this purpose, or if a previous location of the scales has to be corrected or it is unintentionally moved by an impact thereon, without the need for making modifications to the camera. Moreover, it is not necessary to interrupt dialysis treatment when the camera has a malfunction, if there is only one set of scales in the dialysis ward, but several dialysis units are available, which are provided with cameras. In this case, the patient scales have only to be moved into the visual range of another camera.

It is also advantageous if the image information recording device can be swiveled with respect to its recordable field of view.

This allows users to subdivide e.g. the total visual range of a camera in several areas and directing or focusing the camera in a process-controlled manner to a subarea which is required at the moment, or zooming in on such an area for a close-up view or expanding it for a total view. It is also conceivable that only one camera is used for a plurality of patient scales, wherein the camera for weighing procedures is swiveled back and forth between assigned scales.

It is preferred that the image information recording device can be activated, swiveled, and/or deactivated by gestures of the person situated in the field of view, and/or the beginning, interruption, and/or end of a recording procedure can be controlled by gestures of the person situated in the field of view.

This gives the person to be weighed the opportunity to control the recording process by him/herself, for instance to interrupt it for a longer walk to washrooms, or terminate it if operating staff is not available for some time. It is also possible that a caregiver supporting the person to be weighed can easily control the recording process, without being forced to alter the support of the person. As a whole, gesture-based control is also an efficient way of controlling systems, which compared to a slower control scheme via control keys saves storage space and/or reduces data traffic on the data network when the recording is saved in the data network.

The image information recording device is advantageously arranged to detect and provide a direct image of its visual range at least outside the weighing procedure to be recorded.

With the aid of such a direct image, which is also referred to as a live image for which the recording will not be stored yet, the visual range of the camera can be monitored permanently on a screen for example. This kind of monitoring takes place preferably in real time. This allows users for example to observe if a patient is ready for beginning a weighing procedure or if he/she tries to manipulate the scales and/or the weighing procedure in an undesired way, possibly falsifying the measurement results; said observation may take place at a certain distance to the scales if few members of staff have to care for a larger number of patients and are forced to move around in the dialysis ward.

It is preferred that the weight detection device comprises a storage means which is provided on the patient scales and/or on the image information recording device and arranged to retrievably save image information in the form of at least one video still and/or at least one moving picture sequence, which has/have been recorded by the image information recording device, for the purpose of observation, and a display device which is provided on the patient scales and/or on the image information recording device and arranged to display video stills and/or moving picture sequences recorded by the image information recording device and saved by the storage means.

Through this process, displaying and saving all the operations as a video and/or in images are supported in an advantageous way. Weighing procedures are logged and saved by means of images or as a sequence of images. It is possible to identify the caregiver who has carried out the weighing procedure for example, or manually documented protocols can be tracked and/or reproduced on the basis of recordings which have been saved and are displayed at a later point in time.

Preferably, the display device is arranged to display at least one recorded weighing procedure before the dialysis treatment of the patient and at least one recorded weighing procedure after the dialysis treatment of the patient simultaneously and in a comparable fashion.

To this end, the display device may provide several windows or areas, and recordings from different weighing procedures can be loaded into said windows or areas. By means of a timeline for example, it is possible to display desired parts of corresponding weighing procedures or processes next to each other and to check them in comparable fashion for relevant differences and causes of e.g. inexplicable differences in weight between an earlier and a later weighing procedure. Such a check may be performed in a partly automated or fully automated manner.

A dialysis therapy system for carrying out dialysis treatment by determining a patient's weight at least before the dialysis treatment and/or after the dialysis treatment comprises a weight detection device such as briefly described above, at least one dialysis apparatus, which is connected to a (possibly internal) data network and arranged to carry out the dialysis treatment of a patient; at least one work station, which is connected to the data network and comprises at least a screen and a keyboard, and at least one server unit, which is connected to the data network and holds at least one database comprising data relevant to the dialysis treatment; wherein the at least one image information recording device comprises a field of view directed to the dialysis apparatus and/or the patient scales for visually detecting treatment and/or weighing procedures and is arranged to record the course of the dialysis treatment and/or of the weighing procedure within the field of view in such a manner that the cause of a change in the actual weight occurring in the course of a dialysis therapy can be determined from at least one appropriate recording of at least one weighing procedure.

A dialysis therapy system of this type integrates a number of further networked components in addition to the camera-controlled patient scales, wherein specifically the patient scales and the camera are connected, via the data network, to a monitoring system and/or a data transfer and evaluation system, the latter supporting an automated evaluation of the recordings, possibly by adding further data from a networked database and/or further parameters, with extensions, adaptable components, memory space and/or computational power, and the like. Wheelchairs and the like may be identifiable via a barcode or color coding for example. If the weight of such wheelchairs is deposited in a database, any deviation with respect to the weight or a weight mismatch or offset can be calculated automatically.

Advantageously, with such a dialysis therapy system, at least one image information recording device may be installed in the patient scales or provided in its vicinity so as to have a view onto weighing-relevant and output-relevant portions of the patient scales, and at least one image information recording device may be installed in the dialysis apparatus or in its vicinity so as to have a view onto dialysis-relevant and/or weighing-relevant portions of the dialysis apparatus and/or of the patient scales, at least one of the image information recording devices being arranged for observing a patient and/or for a video conferencing connection to a patient.

This allows, for instance, to record a weighing procedure with one camera and make a video telephone call between the physician in charge and the patient being treated, by means of another camera. This proves to be advantageous if the patient is to be questioned during a weighing procedure and/or a dialysis treatment, such as to give the patient medical instructions or to clear up any differences in weight on the spot, without the physician having to be present in person. In this respect, the dialysis ward can be relieved of requiring the presence of staff, and nevertheless the short-term availability of a physician's participation and decision-making can be provided.

It is preferred that the dialysis therapy system comprises a storage means in the data network, arranged to retrievably save image information in the form of at least one video still and/or at least one moving picture sequence, which has/have been recorded by the image information recording device for the purpose of observation, an evaluation device arranged to carry out automatic evaluation of the recorded image information according to predefinable parameters, and a display device arranged to display video stills and/or moving picture sequences recorded by the image information recording device and saved by the storage means.

This allows users to support displaying, evaluating, and/or saving all the operations as a video and/or in images in an advantageous manner. Weighing procedures are logged and saved by means of images or as a sequence of images. It is possible for example to identify the caregiver who has carried out the weighing procedure, or manually documented protocols can be tracked and/or reproduced on the basis of saved recordings that are displayed at a later point in time. Due to the fact that even the dialysis apparatus comprising at least one camera is integrated into the system, movements can be analyzed in a software-supported manner and food intake can be identified, and the quality management of any measures during a dialysis can be provided and evaluated, for instance if any drugs have been administered and also taken, or it is possible to detect if there were any relevant movements of the patient for which corresponding weighing procedures have been carried out, and if the latter exist in valid form.

Moreover, it is advantageous if the predefinable parameters comprise the weight of a patient, identification of the face of the patient, patient data saved on a patient card, the number and/or point in time of a weighing procedure, barcode, and/or color coding, with the parameters capable of being selected and/or combined for identifying predetermined operations during a dialysis treatment and/or for identifying persons involved in the dialysis treatment.

This is helpful especially if a patient is identified on the basis of a combination of weight and face recognition, or when determining if there is a match between patient card and patient, so as to prevent the patient from being treated on an incorrect basis. Suitable selection and/or combination of the parameters allow users to better target the optimization of weight detection and/or dialysis treatment, for instance in cases where known values, possibly changing from one weighing procedure to the next and/or from one dialysis treatment to the next, such as the weights of occasionally used yet different wheelchairs, can be eliminated from the results by calculation.

### BRIEF DESCRIPTION OF THE DRAWINGS

The invention will be explained below on the basis of a preferred exemplary embodiment with reference to the attached drawings in which:

FIG. 1 shows a simplified illustration of a networked dialysis therapy system comprising networked patient scales and networked recording cameras according to an exemplary embodiment;

FIG. 2 shows a simplified illustration of the course of a weighing procedure of a patient with automatic video recording according to an exemplary embodiment for dialysis treatment;

FIG. 3 shows a simplified illustration of the course of a weighing procedure with video recording according to an exemplary embodiment in cooperation with a connected data monitoring and evaluation system; and

FIG. 4 shows a simplified illustration of the known course of a weighing procedure using the logging of detected data on paper.

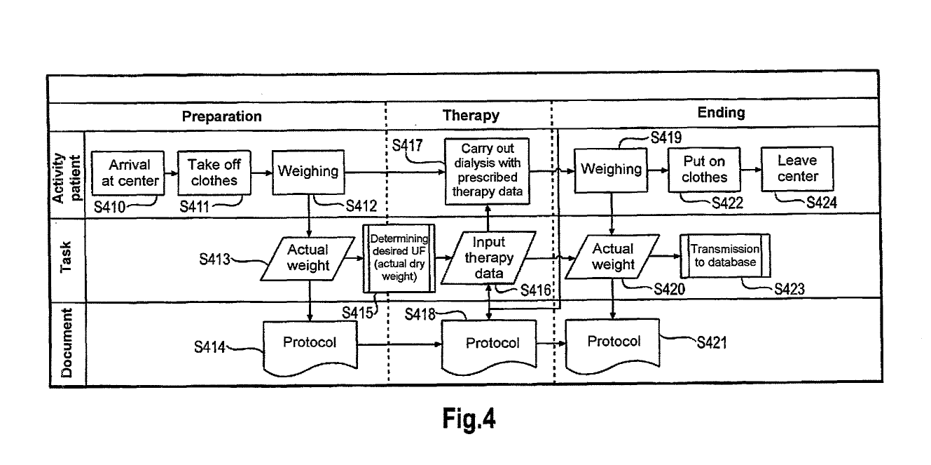

### DETAILED DESCRIPTION OF PREFERRED EMBODIMENTS

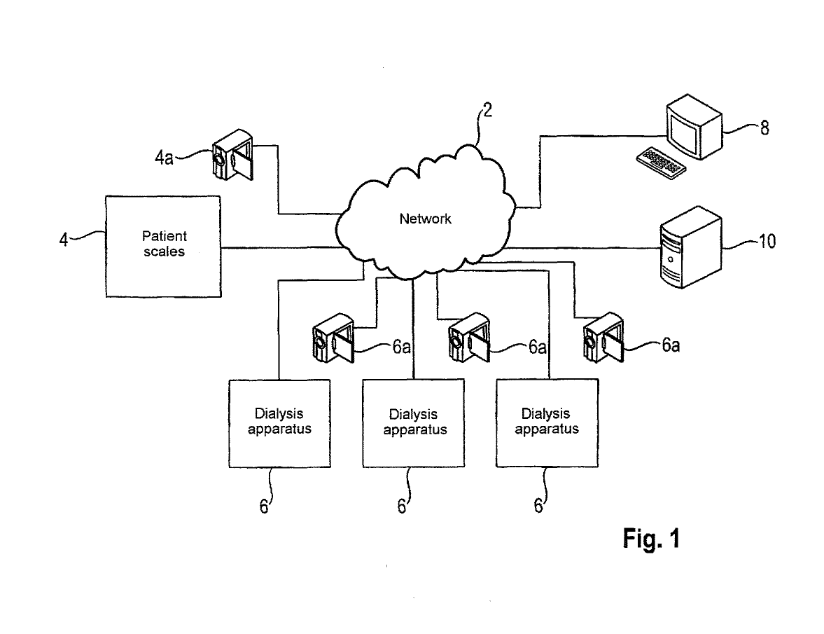

FIG. 1 shows a simplified illustration of a preferably networked weight monitoring system comprising a corresponding, preferably networked weight monitoring device according to an exemplary embodiment. The weight monitoring system and the weight monitoring device are used preferably in a dialysis ward for the dialysis treatment of patients suffering from renal insufficiency. Here, it is preferred that the weight monitoring device used is preferably a set of patient scales of the type known per se, by means of which the weight of a dialysis patient can be determined before, during, and after a dialysis therapy. The weight monitoring system constitutes at least part of a preferably networked overall system, into which the dialysis apparatus itself, as well as various supporting staff-related or physician-related workplaces comprising data stations and/or linked server devices or computers can be integrated.

Specifically, FIG. 1 shows exemplarily, but not limited thereto, a network or data network 2 connecting the various apparatuses and devices to one another, which are integrated into the overall system. The network 2 is not limited to a specific type of network or type of apparatuses networked by same, and may be for instance a known local network (LAN), a wireless network (WLAN), a cell phone network, a combination thereof, or common cabling. Likewise, the kind of data which can be transmitted via the network 2 is not limited to a certain data form. In particular, image data and video data as well as data required for the dialysis treatment can be transmitted and received in an optional direction via the network 2. In addition, the network 2 may provide various interfaces and junctions, for instance access to the Internet, to the intranet of a facility in whose assignment area the dialysis ward is provided, or to a wide area network (WAN) of any other kind, such as to a structure networking various hospitals, research institutions, universities, labs, and the like.

At least one set of patient scales 4 constitutes a weight determination device or weight detection device in the network 2. The basic embodiment and weighing functions of the patient scales 4 are known per se and are not limited to a specific form and/or function either.

FIG. 1 further shows at least one dialysis apparatus 6 capable of carrying out the dialysis treatment of a patient. Three networked dialysis apparatuses 6 are exemplarily illustrated in the network 2.

Furthermore, at least one work or data station 8 comprising at least one keyboard and a screen, such as a client computer or terminal, which may be situated in a staff-related or physician-related workplace, and at least one server computer 10 which may provide a database and/or software and computational power for the work station 8, may be provided in the network 2 and linked with one another.

Reference numeral 4a designates at least one first image capture device or image information recording device, thus at least one first camera. In this exemplary embodiment, the camera 4a is a network-compatible network video camera, i.e. a camera which is connected to the network 2 and designed for recording still and/or moving images within a recording or detection area, e.g. of a lens or an image sensor of the camera, as well as for forwarding them to other receiving and/or processing means in the network 2. In this arrangement, the camera 4a can be arranged for producing and/or processing analog and/or digital image data, yet wherein it is preferred that it produces and uses digital image data.

The first camera 4a may be arranged either directly on the patient scales 4 or separate therefrom in a part of the room in a predetermined position. In particular, the camera 4a is arranged such that its recording area covers any operations on the patient scales 4 which are relevant to the dialysis therapy, such as the standing position of the patient on the scales, externally visible features of the patient, such as his/her clothes and shoes, the area in the vicinity of the patient scales 4, in which the patient may possibly support himself/herself, data displayed by the scales, such as the determined weight of the patient, and/or the course of the weighing procedure, for instance if and how the patient moves, and the like, and is able to detect these. In other words, the at least one camera 4a is arranged and aligned such that it is capable of observing the patient scales 4.

Reference numeral 6a designates at least one second image capture device or image information recording device, thus a second camera. In this exemplary embodiment, the second camera 6a likewise is a network-compatible network video camera, i.e. a camera which is connected to the network 2 and designed for recording still and/or moving images within a recording or detection area, e.g. of a lens or image sensor of the camera, as well as for forwarding them to other receiving and/or processing means in the network 2. In this arrangement, the camera 6a can be arranged for processing analog and/or digital image data, yet wherein using digital image data is preferred.

The camera 6a may be arranged either directly on the dialysis apparatus 6 or separate therefrom in a part of the room in a predetermined position. It is possible to provide one dedicated camera 6a for each of a plurality of dialysis apparatuses 6 or one camera 6a for several dialysis apparatuses 6. In particular, the camera 6a is arranged such that its recording area covers any operations on the dialysis apparatus 6, which are relevant to the dialysis therapy, such as the condition or behavior of a patient connected to the apparatus, externally visible features of the patient, such as his/her clothes and shoes, the surrounding area of the dialysis apparatus 6, data displayed by it, and/or the progress of the dialysis procedure and the like, and is able to detect these. In other words, the at least one second camera 6a is arranged and aligned such that it is capable of observing one or more dialysis apparatuses 6.

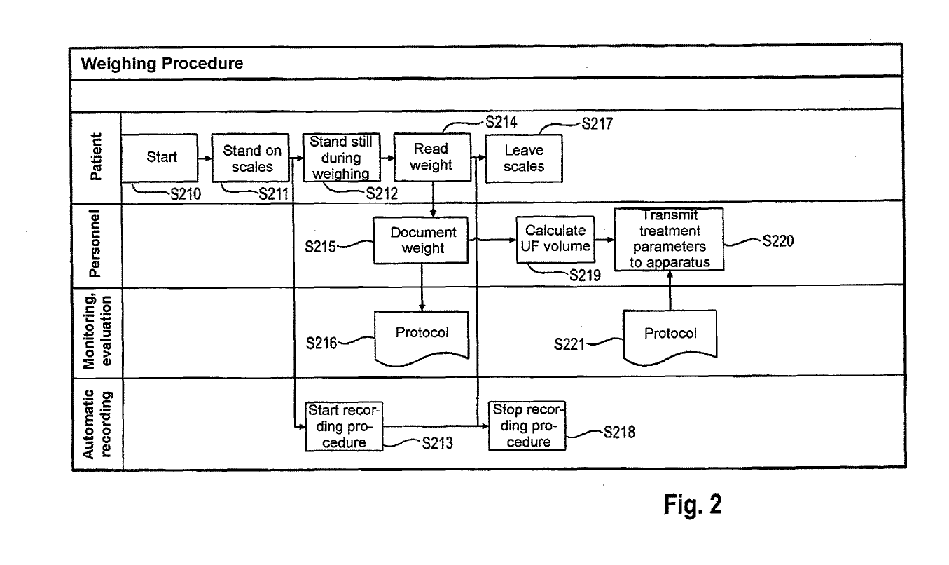

The time schedule of a weighing procedure with automatic image and/or video recording according to an exemplary embodiment for the dialysis treatment will be explained in more detail below. FIG. 2 shows a simplified illustration of such a schedule, in parts and on the basis of steps or activities to be carried out before the dialysis treatment. The term “in parts” means that corresponding steps and activities may also occur during the dialysis treatment after the preparation period and may likewise follow after its end.

It is to be noted that the weighing procedure shown in FIG. 2 may be controlled in an entirely manual fashion; alternatively, it may also proceed in an at least partially automated manner. Specifically, the automatic image and/or video recording process can be activated and/or terminated manually or in an event-driven way, for instance on the basis of signal transmissions from the patient scales 4 or based on mechanisms for detecting gestures, such as pointing and/or waving motions of a predetermined kind and/or direction, whereas the recording, transmitting, and forwarding of produced image data and/or video data via the network 2 to a downstream processing means, e.g. the server 10 or the work station 8, and a local preparation of the data can be performed in automated fashion.

The course shown in FIG. 2 illustrates steps or activities in chronological succession and in excerpts, which, during the procedure of weighing a patient, for instance a dialysis patient with renal insufficiency, are to be carried out in the context of a therapy process including a preliminary weighing procedure before the therapy by the patient and staff of a dialysis ward in this example. Insofar as activities and/or steps have to be performed in parallel and/or simultaneously, the numbering of the individual steps is not necessarily to be understood as a sequence or succession, but for the sake of convenience may only represent an indication of reference numerals for clear assignment.

In step S210, the preliminary process begins with a patient arriving at the dialysis ward and carrying out measures which prepare for the weighing procedure, such as taking off his/her clothes and shoes, and removing bags and the like. In step S211, the patient steps onto the patient scales 4 or takes a seat thereon. If required, said procedure can be supported in a helping manner by staff on the dialysis ward. When the patient is situated on the patient scales 4, the weighing procedure is initiated. This may be effected by the patient himself/herself, by the staff, or in an automatic fashion. “Automatic” for instance in that the first camera 4a responsible for the patient scales 4 already detects the patient on the patient scales 4 in the nature of a direct or live image, without already recording image data, and activates the weighing procedure and/or its recording, for instance as a response to detecting a gesture by the patient or the staff, which can be interpreted as a “start” or “ready” signal. As simultaneously with the activation of the weighing procedure as possible, a recording of the image signals and/or image data generated and delivered by the camera 4a is started in step S213. Such a recording process lasts, preferably in continuous manner, until a defined end of the weighing procedure is signaled and/or detected. While recording the weighing procedure continues, the patient is expected in step S212 to stand still during the weighing process at least for a predetermined period with respect to a reliable and exact weighing result. Following the end of the actual procedure of weighing the patient, which may be signaled by the patient scales 4 after the expiry of a predetermined, sufficiently long period for example, the determined or detected weight is read out from or read on the scales in step S214, and is documented by the staff in step S215, for instance logged in a protocol, in step S216. In step S218, the recording process performed by the camera 4a is stopped or terminated, and in step S217 the patient is then allowed to leave the patient scales 4. Then, in step S219, a member of staff determines the ultrafiltration volume or the ultafiltration quantity from the weight documented in step S215 and in step S220 transfers it to the dialysis apparatus 6, as one of several treatment parameters for the dialysis treatment. In step S221, required treatment parameters may also be taken from the protocol or read out from it by the staff.

Due to the fact that the weighing procedure on the patient scales 4 is monitored, recorded and, for the purpose of later reproduction, and/or further processing, saved by the camera 4a, the entire relevant weighing procedure is documented additionally via a second protocol path, independently of the activities of the staff. If deviations or mismatches are ascertained at a certain point in time, which need to be checked, it is possible to resort to the additional documentation provided by the recording.

The camera can be aligned for instance in such a manner that an image or a recording of the patient is provided during the period when he/she steps onto the scales. With the aid of the recording, it is possible to comprehend or verify whether the patient had put off his/her clothes, jacket, shoes and the like according to directions, before the weighing procedure started. Several temporally spaced recordings may deliver information on whether two weighing procedures which are or can be linked with each other have occurred under different conditions. If for instance wearing shoes is admissible during the weighing procedure, it is possible to find out if a weighing procedure has been carried out with heavy shoes and another one with light shoes.

If the recording by the camera is provided with a date stamp and/or time stamp, any deviations due to the time of day, such as a weighing procedure in the morning and another one in the evening, can be determined.

Further, any reading errors by the staff can be determined using the recording by means of the camera 4a. If during the documentation of the patient's weight the camera is aligned such that an indication, for instance a weight indication in kilograms on a display device of the patient scales 4 or a swing or a balanced state on a dial of the patient scales is recorded, it is possible to find out afterward if a member of staff has read the reading correctly in step S215 and/or the reading has been correctly transferred to the protocol in step S216. If a recording of the protocol, for instance as a video sequence or video still, is provided in addition, it is possible to check in step S220 if a treatment parameter taken from the protocol and transferred to the dialysis apparatus 6 has been read correctly.

In order to integrate such differing aspects of the weighing procedure into a record scheme, provision can be made for the camera 4a or a recording part thereof to be swiveled manually, and/or in a motor-powered manner and/or can be adjusted between close-up adjustment, remote adjustment or tele adjustment. To this end, provision can be made for tracking the camera 4a in accordance with a time schedule of the weighing procedure so as to correspond to the latter, for example by program routines of camera control, which are responsive to gesture-based control, automatically advancing and/or switching in the nature of a switchgear, or on the basis of instructions delivered to the camera via the network 2.

With a manual change of state of the camera 4a, dividing the weighing procedure in predetermined sections, such as “beginning of weighing”, “reading”, and the like, is also conceivable, also coupling the patient scales 4 to the camera 4a such that the weighing procedure, upon reaching the end of a section, cannot be continued until the camera 4a has been changed to a new orientation or adjustment. Exemplarily, provision can be made for the patient scales 4 not to show a detected value which is to be output, until it has registered a confirmation signal or a confirming activity by the staff, to the effect that the camera 4a has been aligned for recording the display or the displayed value and is ready.

In this respect, the process of recording according to FIG. 2 is carried out basically at least for one of the following: documentation, review, traceability, and, as the recording of the weighing procedure is stopped preferably before transmitting treatment parameters to the dialysis apparatus 6, possibly the correction of such treatment parameters of relevant portions of the procedure of weighing a patient on the patient scales 4. If necessary, the transmission of the treatment parameters may also be recorded. To this end, the recording may be started again after step S218 and terminated again following step S220 if the recording area of the camera 4a also includes a treatment parameter input environment or the camera 4a has been correspondingly aligned in advance. As an alternative, the transmission of the treatment parameters to the dialysis apparatus 6 may also be recorded by the camera 6a provided there.

The storage location of the recording of the camera 4a is not limited to a specific one. Saving the recording may take place e.g. locally in the camera 4a itself, for instance on a data carrier connected to the camera 4a such as an exchangeable memory card, an internal data carrier or a data carrier capable of being connected from outside. A copy or backup of the recording can be made at any time via the network 2 on an additional storage location. Alternatively, saving the recording may also be accomplished directly by transmitting the produced image data and/or video data via the network 2 to a predetermined storage means, for example if a camera 4a is used which does not possess any memory means. Alternatively, saving the recording may also take place in a set of patient scales 4 which is provided with a storage means and/or a display device arranged to play back the recording, and is designed in an appropriate manner for this purpose. Here it is advantageous that a weighing procedure and its documentation are available in image and video form on the spot, so that a quick check can be made, if necessary, while the patient is still near the scales for example and weighing conditions have not changed substantially, perhaps due to an interim intake of food and the like, and a weighing procedure can be repeated in very short time if need be.

The time schedule of a weighing procedure with automatic image and/or video recording according to the exemplary embodiment for dialysis treatment will be explained in more detail below in cooperation with a connected data transfer and evaluation system.

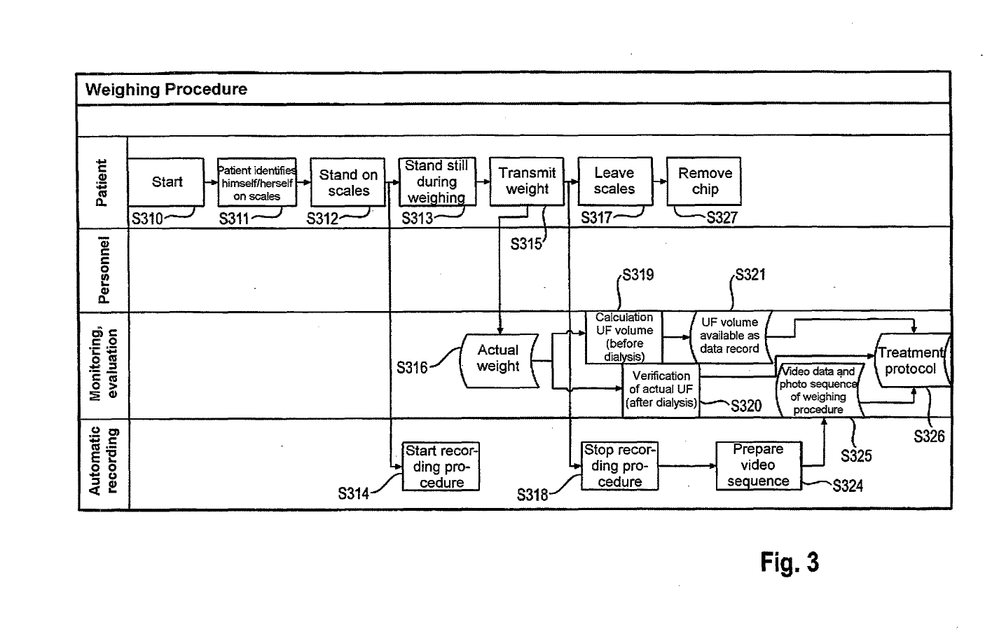

FIG. 3 shows a simplified illustration of such a process in parts and on the basis of steps or activities to be carried out prior to the dialysis treatment. The term “in parts” means that corresponding steps and activities may also occur during the dialysis treatment after the preparation period and may also follow after its end.

It is to be noted that the weighing procedure shown in FIG. 3 may be controlled in an entirely manual fashion; alternatively, it may also proceed in partially automated manner up to and including fully automated manner. Specifically, the automatic image and/or video recording procedure can be activated and/or terminated manually or in an event-driven way, for instance on the basis of signal transmissions of the patient scales 4 or based on mechanisms for gesture detection, whereas the recording itself, and the transmitting and forwarding of produced image data and/or video data via the network 2 to a downstream processing means, e.g. the server 10 or the work station 8, and local preparation of the data can be performed there in automated fashion.

The course shown in FIG. 3 indicates steps or activities in chronological succession and in excerpts, which, during the procedure of weighing the patient, for instance a dialysis patient with renal insufficiency, are to be carried out in the context of a therapy process with a preliminary weighing procedure before the therapy, by the patient and staff of a (in this exemplary case) dialysis ward. Insofar as activities and/or steps have to be performed in parallel and/or simultaneously, the numbering of the individual steps is not necessarily to be understood as a sequence or succession, but for the sake of convenience may only represent an indication of reference numerals for clear assignment.

The data transfer and evaluation system, which is shown in FIG. 3 and is in particular to be seen as a cooperating system, may be software connected via the network 2 and a database for a transparent mapping and for controlling the operations of a dialysis treatment. A system of the applicant, offering basic approaches and functions, is well known from the prior art and may be installed in the work station 8 and/or in the server 10 or be accessible otherwise via these.

Basically, such a data transfer and evaluation system takes data from devices connected to it, such as the weights determined by the patient scales 4, from (not shown) analyzers, during a monitoring operational procedure, and further operates a database providing access to data and entries of e.g. hospital information systems, labs, administration departments, external clinics and physicians, and the like. Depending on their respective functionality, it is possible to implement a unidirectional or bidirectional data exchange between connected devices, data transfer/data detection/data monitoring, the database, work stations or input devices and suppliers, and/or users of the data in the database.

FIG. 3 illustrates the basic functions and tasks in the “monitoring, evaluation” line, which according to the exemplary embodiment are taken over by the data transfer and evaluation system after incorporation of the data from the patient scales 4 in step S316 and after the recording and preparation of the weighing procedure in steps S314, S318 and S324. The weighing procedure and the recording process are carried out as described above with reference to FIG. 2. The functions of the camera 4a are equal to those as described with reference to FIG. 2 except for the differences set forth below.

Specifically, the course according to FIG. 3 begins in step S310. In step S311, the patient proves his/her identity on the patient scales 4 for instance by means of a chip card, a patient card or the like, which is inserted in a card reader by him/her or by the staff. The data transfer and evaluation system identifies the patient on the basis of his/her authentication and supplies already present patient data from the database, or can create a new patient entry using the data saved on the card. In step S312, the patient steps onto the patient scales 4 or takes a seat thereon. Here, step S312 corresponds to step S211 in FIG. 2. In step S313, corresponding to step S212 in FIG. 2, and step S314, corresponding to S213 in FIG. 2, the weighing procedure is carried out and the process of recording it starts. In step S315, other than in FIG. 2, the weight of the patient measured or determined by the patient scales 4, in this exemplary embodiment the actual weight, is not read on the patient scales 4, but transferred to the data transfer and evaluation system. It goes without saying that an additional and readable display of the weight may be provided on the patient scales 4. According to FIG. 3, however, the weight is immediately incorporated into the system and can be represented and read on a screen, preferably in at least nearly real time. Due to its direct incorporation into the system, the weight is documented instantaneously and logged in a corresponding manner, so that in FIG. 3 the corresponding steps in FIG. 2 may be omitted. In step S317, corresponding to step S217 in FIG. 2, the patient leaves the patient scales 4 and in step S327 removes his/her card identifying him/her or lets a member of staff do this, and the process of recording the weighing procedure is terminated in step S318, which corresponds to step S218 in FIG. 2.

Following step S316, in which the actual weight of the patient determined by the weighing procedure is available, the data transfer and evaluation system splits its further processing into a first processing path consisting of steps S319 and S321 and a second processing path consisting of step S320.

In step S319, an ultrafiltration volume or ultrafiltration quantity before dialysis is calculated starting from the actual weight of the patient obtained in step S316. In step S321, said calculated ultrafiltration quantity is made available as a data record for the dialysis treatment, which is not shown in more detail here.

In step S320, the data transfer and evaluation system verifies the ultrafiltration quantity after the end of the dialysis treatment. The actually required ultrafiltration quantity is known after the dialysis treatment and can be compared with the ultrafiltration quantity calculated before the dialysis treatment, on the basis of the actual weight of the patient. Any deviations which are not negligible can be immediately determined in this way.

As simultaneously with or parallel to the steps S319 to S321 as possible, the recorded image sequence and/or video sequence is prepared in an appropriate manner in step S324 after the recording process has been stopped in step S318, for instance said sequence is provided with a time stamp, changed in its format, or compressed, and/or is divided in video data sequences and still or photo sequences of the weighing procedure or of predetermined portions of the latter, which can be utilized by the data transfer and evaluation system. In step S325, the data transfer and evaluation system accepts the image sequence and/or video sequence prepared in step S324.

Subsequently, in step S326, the data transfer and evaluation system creates a treatment protocol of the preceding dialysis treatment. At least the data record from step S321 containing the pre-calculated ultrafiltration quantity, the verification of the ultrafiltration quantity after the dialysis treatment according to step S320, for instance as a result of a comparison or as an absolute value, and the video data and/or image sequence or photo sequence of the weighing procedure are incorporated into the treatment protocol in step S326. In doing so, a link to the image sequences and video sequences saved in another place can be entered into the treatment protocol for example, via which the recording of the weighing procedure can be retrieved if need be.

In association with the positively identified patient, recordings of corresponding weighing procedures can be made available, which have been produced over several treatments, i.e. days or weeks. Recording the weighing procedures by means of the camera 4a thus also allows users to reproduce the individual operations in the longer term.

To this end, provision can be made for the data transfer and monitoring system to represent image data and video data in the form of at least two windows arranged side by side, which show e.g. video stills or video sequences in each case, with the option of the windows each displaying data of one and the same weighing procedure independently of each other, or of two temporally spaced weighing procedures. This allows users to monitor the weighing procedure even if, in a first weighing procedure, the system has already detected a deviation in the actual weight from the actual weight in previous periods in time. In this way, any causes, such as wearing heavy shoes instead of light ones, a jacket which has not been taken off, a bag which has been weighed in addition, the patient supporting him/herself during the weighing process, and the like can be identified. Further, if there are deviations which do not occur for instance until a second weighing procedure, questioning the patient may possibly be omitted, as the weighing procedures can be reproduced by means of the system even without such questioning. Different clothes in the two weighing procedures, different shoes in the two weighing procedures, or baggage in one or two weighing procedures can be identified as the causes of deviations only after the second weighing procedure.

Thus, a weight detection device for determining a patient's weight for dialysis has been described, comprising: patient scales 4 arranged to carry out a weighing procedure and thus detect the actual weight of a patient situated on the scales, a network connection arranged to connect at least one component of the weight detection device, generating information and/or data related to the determining of the patient's weight and/or to the weighing procedure, to a data network 2, an output device arranged to output the actual weight of the patient detected on the patient scales 4 in a readable manner and/or in a predetermined data format to the data network 2, and at least one image information recording device 4a, 6a which comprises a field of view directed to the patient scales 4 for visual detection of weighing procedures and is arranged to record the course of the weighing procedure within the field of view in such a manner that a cause of a change in the actual weight occurring in the course of dialysis therapy can be determined from at least one appropriate recording of at least one weighing procedure. A networked dialysis therapy system or dialysis treatment system comprises a weight detection device of this type, wherein therapeutic aspects, methods, measures and applications may be present within the therapy system, or are to be seen as existing outside the previously described means or device for weight determination with accompanying image and video recording of the weighing procedure.

1. A weight detection device for determining a patient's weight, for dialysis treatment, comprising:
patient scales adapted to carry out a weighing procedure to detect the actual weight of a patient standing on the scales;
an output device adapted to output the actual weight of the patient detected on the patient scales in a readable manner and/or in a predetermined data format to a data network; and
at least one image information recording device which comprises a field of view directed to the patient scales for the visual detection of weighing procedures and is adapted to record the course of a weighing procedure within the field of view in such a manner that the cause of a change in the actual weight occurring in the course of dialysis therapy can be determined from at least one appropriate recording of at least one weighing procedure.

2. The weight detection device according to claim 1, further comprising:
a network connection arranged to connect at least one component of the weight detection device, generating information and/or data related to determining the patient's weight and/or to the weighing procedure, to the data network.

3. The weight detection device according to claim 1, wherein the image information recording device comprises a camera which includes a connection to the data network and is installed in the patient scales.

4. The weight detection device according to claim 2, wherein the image information recording device comprises a camera which includes a connection to the data network and is installed in the patient scales.

5. The weight detection device according to claim 1, wherein the image information recording device comprises a camera including a connection to the data network and/or to the patient scales, said camera being arranged on a dialysis apparatus preferably outside the patient scales.

6. The weight detection device according to claim 2, wherein the image information recording device comprises a camera including a connection to the data network and/or to the patient scales, said camera being arranged on a dialysis apparatus preferably outside the patient scales.

7. The weight detection device according to claim 1, wherein the image information recording device can be swiveled with respect to its recordable field of view.

8. The weight detection device according to claim 1, wherein the image information recording device can be activated, swiveled, and/or deactivated by gestures of a person situated in the field of view, and/or the beginning, interruption and/or end of a recording procedure can be controlled by gestures of a person situated in the field of view.

9. The weight detection device according to claim 1, wherein the image information recording device is arranged to detect and provide a direct image of its visual range at least outside the weighing procedure to be recorded.

10. The weight detection device according to claim 1, further comprising:
a storage means which is provided on the patient scales and/or on the image information recording device and adapted to retrievably save image information in the form of at least one video still and/or at least one moving picture sequence, which has/have been recorded by the image information recording device, for the purpose of observation; and
a display device which is provided on the patient scales and/or on the image information recording device and adapted to display video stills and/or moving picture sequences recorded by the image information recording device and saved by the storage means.

11. The weight detection device according to claim 10, wherein the display device is adapted to display at least one recorded weighing procedure before the dialysis treatment of the patient and at least one recorded weighing procedure after the dialysis treatment of the patient simultaneously and in a comparable fashion.

12. A dialysis therapy system for carrying out dialysis treatment by determining a patient's weight at least before the dialysis treatment and/or after the dialysis treatment, comprising:
a weight detection device according to claim 1;
at least one dialysis apparatus which is connected to the data network and adapted to carry out the dialysis treatment of the patient;
at least one work station which is connected to the data network and comprises at least a screen and a keyboard;
at least one server unit which is connected to the data network and holds at least one database comprising data relevant to the dialysis treatment; wherein
the at least one image information recording device comprises a field of view directed to the dialysis apparatus and/or the patient scales for visually detecting treatment and/or weighing procedures and is adapted to record the course of the dialysis treatment and/or of the weighing procedure within the field of view in such a manner that the cause of a change in the actual weight occurring in the course of the dialysis therapy can be determined from at least one appropriate recording of at least one weighing procedure.

13. The dialysis therapy system according to claim 12, wherein at least one image information recording device is installed in the patient scales or provided in its vicinity so as to have a view onto weighing-relevant and output-relevant portions of the patient scales, and at least one image information recording device is installed in the dialysis apparatus or provided in its vicinity so as to have a view onto dialysis-relevant and/or weighing-relevant portions of the dialysis apparatus and/or of the patient scales, at least one of the image information recording devices being adapted for observing the patient and/or for a video conferencing connection to the patient.

14. The dialysis therapy system according to claim 12, further comprising:
a storage means provided in the data network and adapted to retrievably save image information in the form of at least one video still and/or at least one moving picture sequence, which has/have been recorded by the image information recording device, for the purpose of observation;
an evaluation device arranged to carry out automatic evaluation of the recorded image information according to predefinable parameters; and
a display device adapted to display video stills and/or moving picture sequences recorded by the image information recording device and saved by the storage means.

15. The dialysis therapy system according to claim 13, further comprising:
a storage means provided in the data network and adapted to retrievably save image information in the form of at least one video still and/or at least one moving picture sequence, which has/have been recorded by the image information recording device, for the purpose of observation;
an evaluation device arranged to carry out automatic evaluation of the recorded image information according to predefinable parameters; and
a display device adapted to display video stills and/or moving picture sequences recorded by the image information recording device and saved by the storage means.

16. The dialysis therapy system according to claim 14, wherein the predefinable parameters comprise the weight of the patient, identification of the face of the patient, patient data saved on a patient card, number and/or point in time of a weighing procedure, a barcode, and/or a color coding, with the option of the parameters being capable of being selected and/or combined for identifying predetermined operations during the dialysis treatment and/or for identifying persons involved in the dialysis treatment.

17. The dialysis therapy system according to claim 15, wherein the predefinable parameters comprise the weight of the patient, identification of the face of the patient, patient data saved on a patient card, number and/or point in time of a weighing procedure, a barcode, and/or a color coding, with the option of the parameters being capable of being selected and/or combined for identifying predetermined operations during the dialysis treatment and/or for identifying persons involved in the dialysis treatment.
